(19) United States
(12) Patent Application Publication
(10) Pub. No.: US 2010/0036190 A1
(43) Pub. Date: Feb. 11, 2010

(54) INTERNAL RADIATION SHIELD FOR BRACHYTHERAPY TREATMENT

(76) Inventors: Brent D. Murphy, South Bend, IN (US); James Wheeler, Goshen, IN (US)

Correspondence Address: BARNES & THORNBURG LLP, 11 SOUTH MERIDIAN, INDIANAPOLIS, IN, 46204, US

(21) Appl. No.: 12/488,972

(22) Filed: Jun. 22, 2009

Related U.S. Application Data
(60) Provisional application No. 61/075,196, filed on Jun. 24, 2008.

(51) Int. Cl. A61M 36/04

(52) U.S. Cl. 600/7

(57) ABSTRACT

A brachytherapy shielding system configured to be used with a brachytherapy application includes a balloon catheter and a body made from a magnetic material positioned within an inflatable portion, such as a balloon portion, of the balloon catheter. The brachytherapy shielding system is arranged to shield skin tissue exposed to a radiation source during intracavitary radiation therapy.

This application claims priority under 35 U.S.C. §119(e) to U.S. Provisional Patent Application Ser. No. 61/075,196, filed Jun. 24, 2008 entitled INTERNAL RADIATION SHIELD FOR BRACHYTHERAPY TREATMENT, the entirety of which is hereby incorporated by reference herein.

### CROSS REFERENCE TO RELATED APPLICATIONS

Cross reference is made to U.S. application Ser. No. 11/877,424 titled BRACHYTHERAPY SHIELDING SYSTEM, which was filed on Oct. 23, 2007 by Brent D. Murphy and James Wheeler, was assigned to the same assignee as the present application, and is hereby incorporated by reference herein.

### BACKGROUND

The present disclosure relates to brachytherapy and particularly to a system for shielding a patient from the intracavitary radiation emitted during brachytherapy sessions.

In some instances, the ability to perform intracavitary brachytherapy is contingent upon the patient's “skin thickness” adjacent to a lumpectomy cavity. Care providers may be reluctant to provide this procedure when a skin distance (the distance between an inserted brachytherapy applicator and an external skin surface) is less than 7 mm. For example, a small skin distance, such as a skin distance less than 5 mm, may increase the risk of radiation induced burns to the skin tissue adjacent to the radiation source. Illustratively, therefore it may be desirable for the skin distance to be at least 10 mm or more.

### SUMMARY

The present invention may comprise one or more of the features recited in the attached claims, and/or one or more of the following features and combinations thereof.

According to the present disclosure, a brachytherapy shielding system configured to be used with a brachytherapy applicator during brachytherapy treatment includes a balloon catheter and a body made from a magnetic material. The balloon catheter includes a catheter portion and an inflatable portion coupled to the catheter portion and adapted for intracavity implantation into a patient. The body is positioned within the inflatable portion and is coupled to the catheter portion of the balloon catheter.

In one illustrative embodiment, the system may further include a magnetic source configured to be positioned adjacent to an exterior skin surface of the patient adjacent to the balloon to attract the body thereto. Illustratively, the magnetic source may be a neodymium iron boron magnet.

In another illustrative embodiment, the body may be positioned adjacent an outer surface of the catheter portion.

In still another embodiment, the inflatable portion may include a balloon portion and the body may be coupled to a proximal portion of the catheter portion to be positioned within the balloon portion. Further illustratively, the body may include a passageway therethrough such that the proximal portion of the catheter portion is received within the passageway of the body.

In another illustrative embodiment, the body may be generally cylindrical in shape.

In yet another illustrative embodiment, the body may be made from one or more of Samarium Cobalt-5, Samarium, Gold, Iron, Iron Oxide, and Gadolinium. Illustratively, the body may be approximately 1 cm long.

In still another illustrative embodiment, the inflatable portion of the catheter may include a sponge portion.

According to another aspect of the present disclosure, a method for shielding radiation during a brachytherapy treatment includes implanting a balloon catheter in a cavity of a patient, wherein the balloon catheter includes a magnetic material within a balloon portion of the balloon catheter, positioning a magnetic source adjacent an external surface of the patient and adjacent the cavity of the patient to attract the magnetic material thereto, implanting a brachytherapy applicator within the cavity of the patient adjacent the balloon catheter, and filling the balloon portion of the balloon catheter with a liquid.

In one illustrative embodiment, the balloon portion of the balloon catheter may be filled with liquid before the brachytherapy applicator is implanted within the cavity of the patient. Further illustratively, the balloon portion of the balloon catheter may be positioned between a balloon of the brachytherapy applicator and the external skin surface of the patient. In particular, the balloon portion of the balloon catheter may be filled with an amount of liquid sufficient to position a proximal end of a catheter portion of the brachytherapy applicator approximately 7-10 mm away from the external skin surface of the patient.

In another illustrative embodiment, the method further includes imaging the cavity of the patient after implanting both the balloon catheter and the brachytherapy applicator.

According to still another aspect of the present disclosure, a method for shielding radiation during a brachytherapy treatment includes implanting a balloon catheter in a cavity of a patient, implanting a brachytherapy applicator in the cavity of the patient adjacent the balloon catheter, and creating a radiation shield within the patient after implanting the balloon catheter.

In one illustrative embodiment, a radiation attenuating material may be placed within a balloon portion of the balloon catheter. Illustratively, the radiation attenuating material may include a liquid.

In other illustrative embodiments, a magnetic source may be placed adjacent an external skin surface of the patient to attract a magnetic material positioned within a balloon portion of the balloon catheter thereto.

In yet another illustrative embodiment, the balloon catheter may be positioned between the brachytherapy applicator and the external skin surface of the patient.

Additional features of the present disclosure will become apparent to those skilled in the art upon consideration of the following detailed description of illustrative embodiments exemplifying the best mode of carrying out the disclosure as presently perceived.

### BRIEF DESCRIPTION OF THE DRAWINGS

The detailed description particularly refers to the accompany figures in which:

FIG. 1 is a diagrammatic view of a brachytherapy shielding system in accordance with the present disclosure showing a magnetic source and a balloon catheter in a deflated position;

FIG. 2 is a diagrammatic view of the shielding system of FIG. 1 showing the balloon of the catheter filled with water and also showing a sleeve made from a magnetic material coupled to a proximal end of the catheter within the balloon;

FIG. 3 is a diagrammatic view of the shielding system of FIG. 1 showing the balloon catheter inserted into a lumpectorny cavity of a patient and the magnetic source positioned adjacent the external skin surface of the patient to attract the sleeve of the catheter to an upper, interior surface of the cavity;

FIG. 4 is a view similar to FIG. 3 showing the balloon of the catheter filled with water and positioned near the upper, interior surface of the cavity;

FIG. 5 is a view similar to FIG. 4 showing a brachytherapy applicator inserted into the cavity below the balloon catheter to deliver radiation treatment to surrounding tissues;

FIG. 6 is a diagrammatic view of an alternative shielding system of the present disclosure similar to the system shown in FIGS. 1-5 showing a compressed sponge attached to a catheter and a sleeve made from a magnetic material positioned within the sponge to interact with the external magnetic source; and

FIG. 7 is a view similar to FIG. 6 showing the sponge having been “inflated” with water and further showing the brachytherapy applicator delivering radiation treatment to the patient.

### DETAILED DESCRIPTION

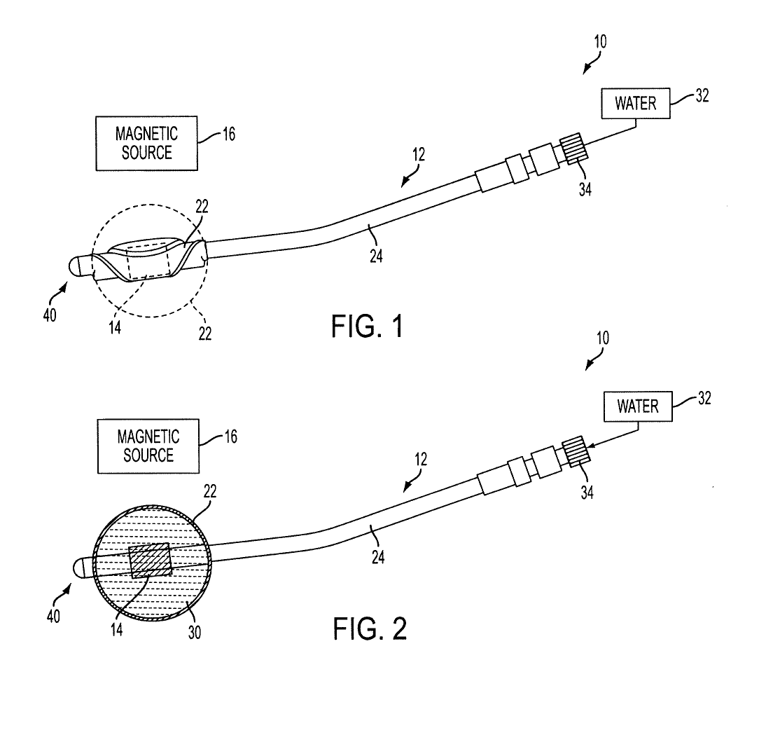

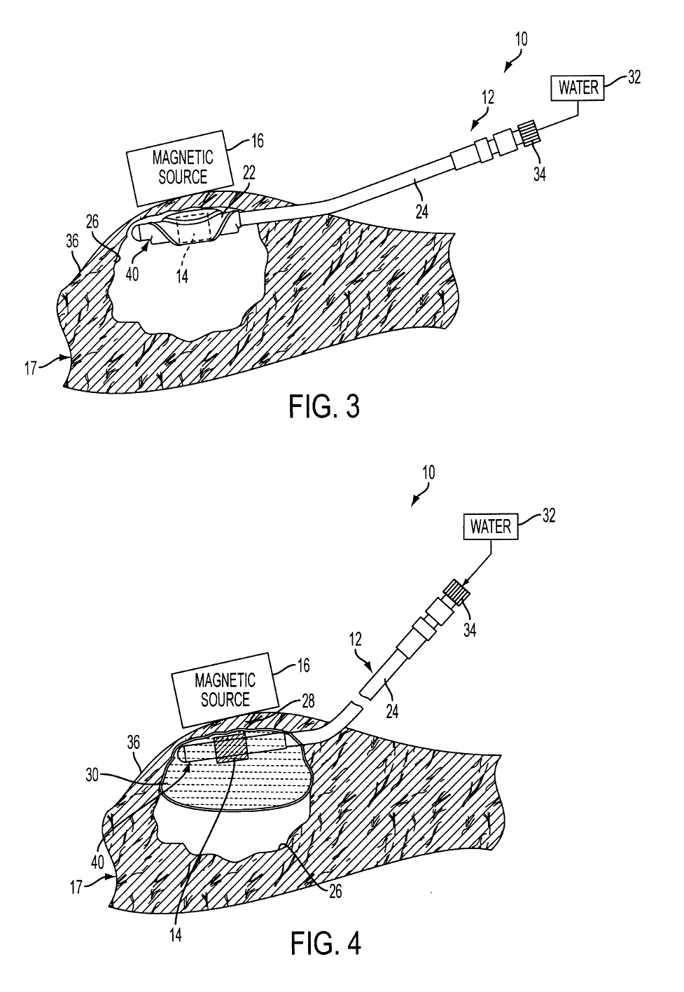

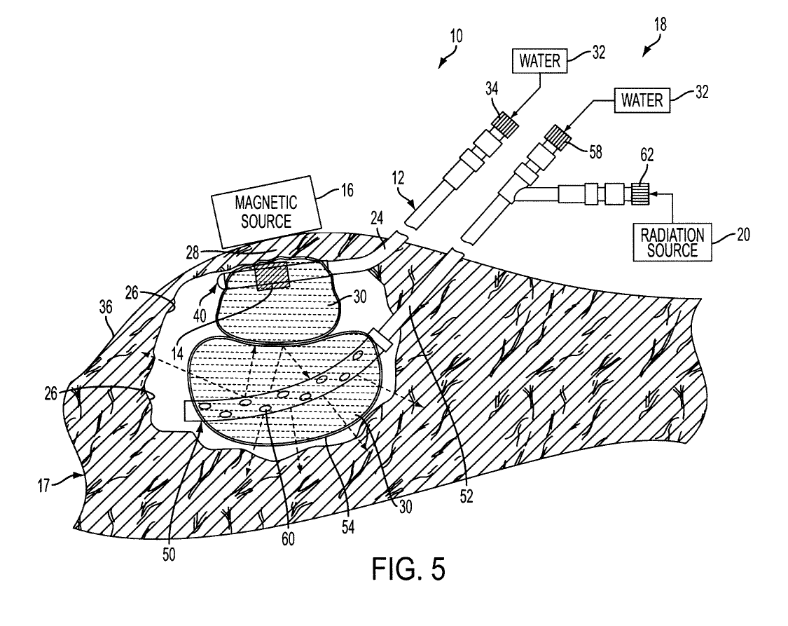

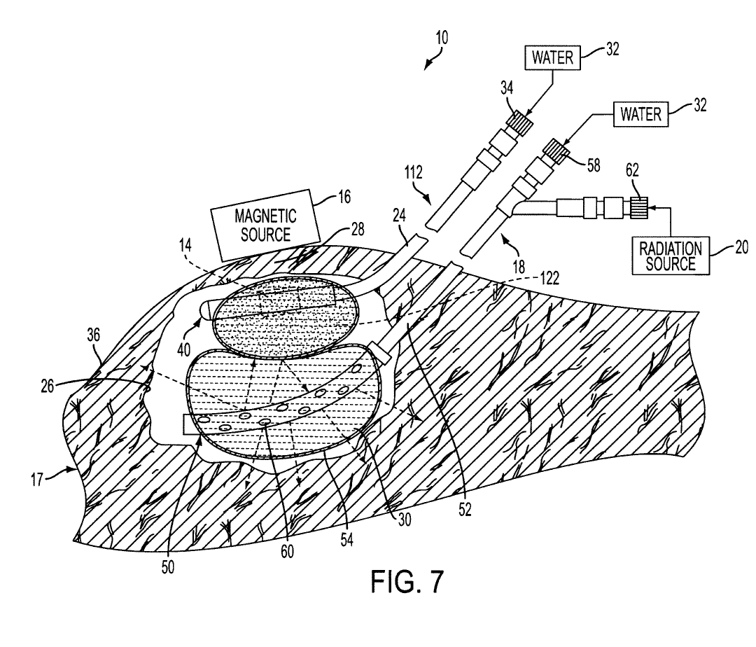

A brachytherapy shielding system 10 includes a balloon catheter 12, a body, or sleeve, 14 made from a magnetic material coupled to the balloon catheter 12, and a magnetic source 16, as shown in FIG. 1. The balloon catheter 12 is adapted for intracavital implantation into a patient 17 as shown in FIGS. 3-7. Illustratively, a brachytherapy applicator 18 (shown in FIGS. 5 and 7) is configured to provide localized radiation therapy to surrounding tissue after a cancerous lump or growth has been removed from the body of the patient such as, for example, in a breast “lumpectomy” or other similar procedure. The shielding system 10 is configured to attenuate a radiation dose emitted by one or more radiation seeds 60 from a radiation source 20 to shield skin tissue 28 of the patient 17 (or other portions of the patient's anatomy) adjacent to the brachytherapy applicator 18. As is discussed in greater detail below, the body 14 and the magnetic source 16 cooperate to position a proximal end 40 of the balloon catheter 12 between the portion 28 of the patient's 17 skin tissue to be shielded and the brachytherapy applicator 18 in order to shield the skin tissue 28 from radiation delivered by the brachytherapy applicator 18.

Illustratively, the balloon catheter 12 is a cavity evaluation device (CED), such as a MammoSite® CED available from Cytyc Corporation of Marlborough, Mass. However, it is within the scope of this disclosure to use another suitable CED as well. Further, while the illustrative balloon catheter 12 is a CED, it is within the scope of this disclosure for the shielding device 10 to include other balloon catheters suitable for intracavity insertion into a patient's lumpectomy cavity.

The illustrative balloon catheter 12 includes an inflatable balloon portion 22 coupled to a catheter portion 24, as shown in FIG. 1. Illustratively, the balloon portion 22 is coupled to a proximal end 40 of the catheter portion 24. The balloon portion 22 is configured to be placed inside a lumpectomy cavity 26 through a small incision in, for example, the breast of the patient 17 as shown in FIGS. 3-7. The body 14 is also coupled to a proximal end 40 of the catheter portion 24.

Illustratively, the body 14 is generally cylindrical in shape and is positioned around the proximal end 40 of the catheter portion 24 that is located inside the balloon portion 22. Accordingly, the body 14 surrounds a portion of the proximal end 40 of the catheter portion 24 and operates as a sleeve around the catheter portion 24. Further illustratively, the body 14 is enclosed within the balloon portion 22 when the balloon portion 22 is both inflated and deflated, as shown in FIGS. 1 and 2. As stated above, the body 14 is cylindrical in shape and includes a passageway (not shown) configured to receive a portion of the proximal end 40 of the catheter portion 24 therethrough; however, it is within the scope of this disclosure to include a magnetic body having any suitable shape and size coupled to the proximal end 40 of the catheter portion 24. Furthermore, the illustrative body has a length of approximately 1 cm; however, any suitably sized sleeve may be used. In the illustrative embodiment, the magnetic body 14 may include one or more of, but not limited to, Samarium Cobalt-5 (SmCo₅), Samarium, Gold, Iron, Iron Oxide, and Gadolinium, Iron (III) Oxide or any suitable material attracted to a magnetic field. It is within the scope of this disclosure, however, for the body 14 to be made from any suitable material that is capable of being magnetized or that which is attracted to a magnetic source.

The body 14 may be coupled to the proximal end 40 of the catheter portion 24 in any suitable manner. For example, an adhesive may be used to secure the body 14 to the catheter portion 24. Alternatively, a mechanical locking mechanism including tabs and notches formed in each of the catheter portion 24 and the body 14 may be used as well. Further, the body 14 may be coupled to the catheter portion 24 through an interference fit in order to generally prevent movement of the body 14 relative to the catheter portion 24. Of course, it is within the scope of this disclosure to rigidly couple the body 14 to the proximal end 40 of the catheter portion 24 using any number of suitable devices or methods. Further, it is within the scope of this disclosure to couple the body 14 to the proximal end 40 of the catheter portion 24 without the use of any such locking mechanisms. In other words, the proximal end 40 of the catheter portion 24 may simply be received within the passageway of the body 14 in order to retain the body 14 thereon.

Typically, a CED, such as the balloon catheter 12 is used to assess the size and/or shape of the patient's lumpectomy cavity. For example, a care provider may typically use a CED to determine the distance between the proximal end 40 of the catheter portion 24 and the patient's outer skin surface 36 (the skin-to-cavity distance). When used to assess the cavity 26, distal end of a CED, including the uninflated balloon on the distal end of the CED, is inserted into the patient's lumpectomy cavity 26. Once in place, the balloon of the CED is inflated with a saline solution. After evaluation of the cavity 26, the CED is removed and the appropriately-sized brachytherapy applicator may be selected and placed within the cavity 26 to deliver radiation treatment. This assessment may be conducted by using the balloon catheter 12 of the present disclosure including the metal sleeve 14 attached thereto. Illustratively, the balloon catheter 12 having the magnetic body 14 attached thereto may be used for such evaluation purposes as well.

In use as a shield from the radiation emitted during brachytherapy treatments, the proximal end 40 of the catheter portion 24 and the uninflated balloon portion 22 coupled to the proximal end 40 of the catheter portion 24 are inserted into the cavity 26, as shown in FIG. 3. A magnetic source 16 of the shielding device 10 is placed adjacent the exterior surface 36 of the portion 28 of the patient's skin which requires protection or shielding from the radiation treatment. Illustratively, the magnetic source 16 may positioned on or adjacent the patient's skin surface 36 at a location approximately where a skin-to-cavity distance is the smallest. Accordingly, the magnetic source 16 attracts the body 14 on the applicator 12 to position the body 14 adjacent the portion 28 of the patient's skin which requires shielding, as shown in FIG. 3. In illustrative embodiments, the magnetic source 16 is a neodymium iron boron (NdFeB) magnet; however, any magnetic source including permanent magnets and electromagnets, which are suitable for medical use, may be used.

Once the proximal end 40 of the catheter portion 24 is properly placed, the balloon portion 22 is then “inflated” with a liquid, such as sterile water 30 from a water source 32, through a catheter port 34, as shown in FIG. 4. While water 30 is illustratively used to inflate the balloon portion 22, it is within the scope of this disclosure to use any suitable liquid, such as saline solution, for example. Further, it is within the scope of this disclosure to inflate or fill the balloon portion 22 with any suitable radiation attenuating material. As stated above, the balloon portion 22 is inserted into the cavity 26 of the patient. However, a distal portion of the catheter portion 24 remains outside the patient and may be secured to a cushioned gauze pad (not shown) to prevent movement of the catheter portion 24 during the procedure.

Illustratively, the balloon portion 22 is partially-inflated in order to provide room within the cavity 26 for the brachytherapy applicator 18. As is discussed in greater detail below, the water 30 within the balloon portion 22 operates to distance and shield the skin 28 from the radiation emitted from the brachytherapy applicator 18 during the radiation treatment. Accordingly, the balloon portion 22 of the applicator 12 is filled with a suitable amount of water 30 to provide the desired amount of attenuation or shielding of the radiation being emitted. As such, the care provider may adjust the amount of water 30 within the balloon portion 22 as necessary depending upon the size of the cavity 26, the amount of radiation to be emitted, and the distance of the skin 28 from the radiation seeds (not shown) within the brachytherapy applicator 18, for example. Illustratively, the balloon portion 22 is filled with approximately 5-10 mL of water to achieve a suitable distance between the skin 28 and a 7-10 mm sized brachytherapy applicator. Accordingly, the balloon portion 22 is sufficiently filled to position the proximal end 50 of the catheter 52 of the brachytherapy applicator 18 approximately 7-10 mm away from the external skin surface of the patient.

Once the balloon portion 22 is inflated, the brachytherapy applicator 18 is inserted into the cavity 26, as shown in FIG. 5. Illustratively, the applicator 18 is inserted through the same incision (not shown) in the patient's skin as the catheter 12; however, it is within the scope of this disclosure to insert the applicator 18 into the cavity 26 through a separate incision. Further, the brachytherapy applicator 18 may be inserted into the cavity 26 before inflating the balloon portion 22 of the applicator 12. Illustratively, the brachytherapy applicator 18 is positioned below the balloon catheter 12 within the cavity 26 such that the balloon portion 22 of the applicator 12 is located between the skin 28 of the patient 17 and a proximal end 50 of the brachytherapy applicator 18.

Once the proximal end 50 of the catheter 52 of the brachytherapy applicator 18 is inserted into the cavity 26, the balloon 54 of the applicator 18 is “inflated” a liquid, such as water 30 from the source 32 or a saline solution, through a first catheter port 58 such that the balloon 54 fits snugly into the cavity 26 and generally conforms to the size of the cavity 26, as shown in FIG. 5. Typically, approximately 35-70 cc of liquid, such as water or saline solution, is used to inflate or fill an applicator 18 have a 4-5 cm balloon 54 while approximately 70-120 cc of liquid is used to fill an applicator 18 having a 5-6 cm balloon 54. Once the balloon 54 of the brachytherapy applicator 18 has been filled, the magnetic source 16 may be removed. Illustratively, the inflated balloon 54 of the applicator 18 operates to hold the inflated balloon portion 22 of the balloon catheter 12 in place.

Both the balloon portion 22 of the balloon catheter 12 and the balloon 54 of the applicator 18 remain inflated during the duration of the brachytherapy treatment. Initially, the water 30 within each of the balloons 22, 54 may contain a contrast media so that the balloons 22, 54 are “visible” during computed tomography (CT) or other suitable types of imaging of the balloon catheter 12 and applicator 18 within the cavity 26. Such a visualization of the balloons 22, 54 may allow the care provider to determine the size and position of the balloons 22, 54 and make any adjustments necessary.

Once the balloon 54 is inflated and properly positioned within the cavity 26 a portion of the catheter 52 that remains outside the breast is coupled to a computer-controlled High Dose Rate (HDR) machine (not shown) that inserts the radiation seed or seeds 60 through a second catheter port 62 to deliver the radiation therapy. The radiation seeds 60 are deposited through a distal end of catheter 22 into the proximal end 50 of catheter 52 such that the seeds 60 are positioned within the balloon 54 and thus within the cavity 26. In illustrative embodiments, the radiation seeds 60 are Iridium-192; however any suitable radiation source may be used.

As noted above, the body 14 is made from a magnetic material and is, therefore, attracted to the magnetic field of magnetic source 16 thus positioning the water-filled balloon portion 22 between the brachytherapy applicator 18 and the patient's skin 28. Accordingly, the water 30 within the balloon portion 22 operates to attenuate the radiation dose emitted from the seeds 60, as shown in FIG. 5. Further, the magnetic source 16 and the body 14 cooperate to position the shield, i.e., the water 30 within the balloon portion 22, adjacent the portion 28 of the patient's skin to be protected, thereby reducing the amount of radiation received by the portion 28 of the patient's skin or other selected tissue. Illustratively, the position of the magnetic source 16 adjacent the patient 17 may be moved or adjusted relative to the patient 17 in order to move the magnetically-attractable body 14 and the water-filled balloon portion 22 therewith in order to shield a different portion of the patient 17 from the radiation emitted from the seeds 60 within the brachytherapy applicator 18.

Prior to insertion of the radioactive seeds 60, the care provider may desire to determine whether a sufficient amount of water 30 is present within the balloon portion 22 to effectively attenuate the radiation emission of seeds 60 in order to shield the adjacent skin or tissue portion 28 of the patient 17 from the radioactive energy. Thus, as noted above, it may be desirable to subject the patient to CT imaging (or any other suitable imaging) of the balloon catheter 12 and the brachytherapy applicator 18 while one or more of the catheter 12 and the applicator 18 is positioned within the lumpectomy cavity 26. If the care provider determines that an insufficient amount of the water 30 is present within the balloon portion 22, additional saline may be added to the balloon portion 22 to increase the shielding material present. Further, if the care provider determines that the balloon portion 22 containing the water 30 is improperly placed, the care provider may move the magnetic source 16 to adjust the position of the body 14, and thus the balloon portion 22. Illustratively, once the radiation treatment session is finished, the water 30 from within the balloons 22, 54 is withdrawn such that the balloons 24, 54 return to their un-inflated state. The applicator 18 and the balloon catheter 12 may then be removed.

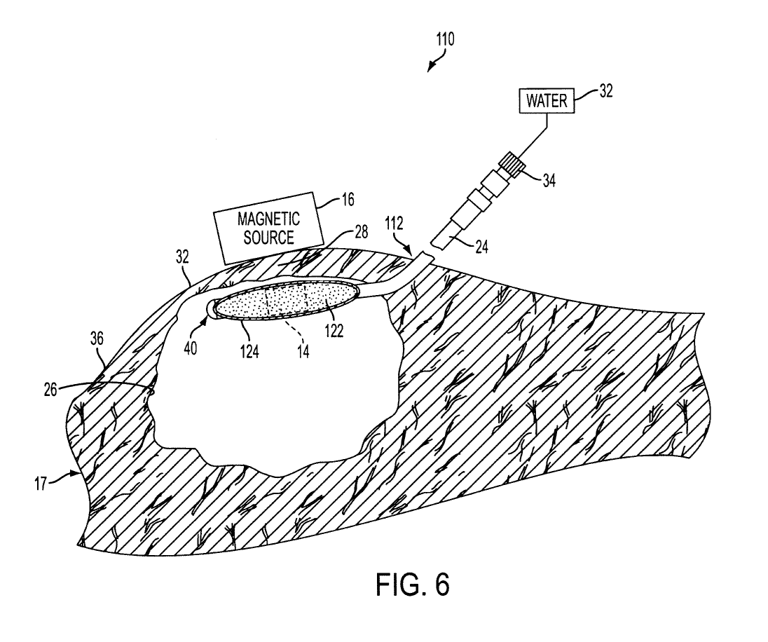

Looking now to FIGS. 6 and 7, an alternative brachytherapy shielding system 110 includes an applicator 112 having the catheter portion 24 and a sponge portion 122 coupled to the proximal end 40 of the catheter portion 24. Illustratively, the sponge 122 is encased within a liquid impermeable enclosure 124. The applicator 112 further includes the body 14 around the proximal end 40 of the catheter portion 24 within the sponge 122. In use, the shielding system 110 operates the same as or similarly to the system 10 such that the sponge 122 is inserted into the cavity 26 while the sponge 122 is in an uninflated, or unexpanded, state, as shown illustratively in FIG. 6. The magnetic source 16 is then used to attract the body 14 thereto and position the sponge 122 in a suitable location determined by the care provider. Once properly positioned, the water 30 is used to expand the sponge 122 to a size suitable for attenuating the radiation emitted by the radiation seeds 60 of the brachytherapy applicator 18, as shown in FIG. 7. Illustratively, therefore the sponge 122 and the water 30 cooperate to create the radiation shield. Accordingly, both the balloon catheters or applicators 12, 112 include the catheter portion 24 and an inflatable portion. In particular, the inflatable portion of the catheter 12 is the balloon portion 22 whereas the inflatable portion of the catheter 112 is the sponge portion 122.

Illustratively, FIGS. 1-7 illustrate the use of the shielding systems 10, 110 for use with the brachytherapy applicators 18 using radiation seeds 60. Illustratively, the applicator 18 is a MammoSite® “balloon catheter” available from Cytyc Corporation of Marlborough, Mass. It is within the scope of this disclosure, however, to use the shielding systems 10, 110 with other suitable brachytherapy applicators as well. For example, the shielding systems 10, 100 may be used with the Axxent® Electronic Brachytherapy System® provided by Xoft, Inc. of Fremont, Calif.

Illustratively, a kit for building a brachytherapy shielding system may be provided. Such a kit may include the magnetic body 14, the magnetic source 16, and an instruction sheet (not shown) providing instructions for creating a radiation shield. Such instruction sheet may include instructions for filling the balloon portion 22 with a recommended amount of water, placement and operation of the magnetic source 16 on the patient, and/or use of an imaging maching to determine the position and thickness of the balloon portion 22 creating the shield, for example.

1. An internal brachytherapy shielding system configured to be used with a brachytherapy applicator during brachytherapy treatment comprising
a balloon catheter having a catheter portion and an inflatable portion coupled to the catheter portion and adapted for intracavity implantation into a patient, and
a body made from a magnetic material positioned within the inflatable portion and coupled to the catheter portion of the balloon catheter.

2. The brachytherapy shielding system of claim 1, further comprising a magnetic source configured to be positioned adjacent to an exterior skin surface of the patient adjacent to the inflatable portion to attract the body thereto.

3. The brachytherapy shielding system of claim 2, wherein the magnetic source is a neodymium iron boron magnet.

4. The brachytherapy shielding system of claim 1, wherein the body is positioned adjacent an outer surface of the catheter portion.

5. The brachytherapy shielding system of claim 4, wherein the body defines a passageway therethrough such that the proximal portion of the catheter portion is received within the passageway of the body.

6. The brachytherapy shielding system of claim 1, wherein the inflatable portion includes a balloon portion and the body is coupled to a proximal portion of the catheter portion and is positioned within the inflatable portion.

7. The brachytherapy shielding system of claim 1, wherein the body is generally cylindrical in shape.

8. The brachytherapy shielding system of claim 1, wherein the body is made from one or more of Samarium Cobalt-5, Samarium, Gold, Iron, Iron Oxide, and Gadolinium.

9. The brachytherapy shielding system of claim 1, wherein the body is approximately 1 cm long.

10. The brachytherapy shielding system of claim 1, wherein the inflatable portion includes a sponge portion.

11. A method for shielding radiation during a brachytherapy treatment, the method comprising
implanting a balloon catheter in a cavity of a patient, wherein the balloon catheter includes a magnetic material within a balloon portion of the balloon catheter,
positioning a magnetic source adjacent an external surface of the patient and adjacent the cavity of the patient to attract the magnetic material thereto,
implanting a brachytherapy applicator within the cavity of the patient adjacent the balloon catheter, and
filling the balloon portion of the balloon catheter with a liquid.

12. The method of claim 11, wherein filling the balloon portion of the balloon catheter with the liquid occurs before implanting the brachytherapy applicator within the cavity of the patient.

13. The method of claim 11, wherein filling the balloon portion of the balloon catheter including filling the balloon portion with an amount of liquid sufficient to position a proximal end of a catheter portion of the brachytherapy applicator approximately 7-10 mm away from the external skin surface of the patient.

14. The method of claim 11, further comprising positioning the balloon portion of the balloon catheter between a balloon of the brachytherapy applicator and the external skin surface of the patient.

15. The method of claim 11, further comprising imaging the cavity of the patient after implanting both the balloon catheter and the brachytherapy applicator.

16. A method for shielding radiation during a brachytherapy treatment, the method comprising
implanting a balloon catheter in a cavity of a patient,
implanting a brachytherapy applicator in the cavity of the patient adjacent the balloon catheter, and
creating a radiation shield within the patient after implanting the balloon catheter.

17. The method of claim 16, wherein creating the radiation shield includes placing a radiation attenuating material within a balloon portion of the balloon catheter.

18. The method of claim 17, the radiation attenuating material includes a liquid.

19. The method of claim 16, wherein creating the radiation shield further includes placing a magnetic source adjacent an external skin surface of the patient to attract a magnetic material positioned within a balloon portion of the balloon catheter thereto.

20. The method of claim 16, further comprising positioning the balloon catheter between the brachytherapy applicator and the external skin surface of the patient.
